(19) United States
(12) Patent Application Publication
(10) Pub. No.: US 2022/0273742 A1
(43) Pub. Date: Sep. 1, 2022

(54) MATERIALS AND METHODS FOR BLOCKING MALARIA INFECTION AND TRANSMISSION

(71) Applicant: JUN LI, MIAMI, FL (US)

(72) Inventor: JUN LI, MIAMI, FL (US)

(73) Assignee: THE FLORIDA INTERNATIONAL UNIVERSITY BOARD OF TRUSTEES, MIAMI, FL (US)

(21) Appl. No.: 17/679,355

(22) Filed: Feb. 24, 2022

Related U.S. Application Data
(60) Provisional application No. 63/152,949, filed on Feb. 24, 2021.

(51) Int. Cl. A61K 36/062; A61K 31/366; A61P 33/06

(52) U.S. Cl. CPC A61K 36/062; A61K 31/366; A61P 33/06

(57) ABSTRACT

The subject invention provides fungal extracts, fungal metabolites, pharmaceutical compositions comprising the fungal extracts, and/or fungal metabolites, methods of preparation, and therapeutic uses thereof. The subject invention also provides a bioactive agent and a composition comprising the bioactive agent, and therapeutic uses thereof. The subject invention further provides methods for treating, inhibiting and/or preventing malaria infection and transmission by using the fungal extracts, fungal metabolites, bioactive agents, and pharmaceutical compositions comprising the fungal extracts, fungal metabolites, and/or bioactive agent.

### CROSS-REFERENCE TO RELATED APPLICATION

This application claims the benefit of U.S. Provisional Application Ser. No. 63/152,954 filed Feb. 24, 2021, which is hereby incorporated by reference herein in its entirety.

### GOVERNMENT SUPPORT

This invention was made with government support under AI125657 awarded by the National Institutes of Health. The government has certain rights in the invention.

### SEQUENCE LISTING

The Sequence Listing for this application is labeled “SeqList-07Feb22-ST25.txt,” which was created on Feb. 7, 2022, and is 1 KB. The Sequence Listing is incorporated herein by reference in its entirety.

### BACKGROUND OF THE INVENTION

Plasmodium parasites transmitted by anopheline mosquitoes caused approximately 200 million clinical malaria cases and half a million deaths in 2019, according to a recent World Health Organization report. Most antimalarial drugs kill the parasites at the blood stage. Since the passage of Plasmodium through vector mosquitoes is a necessary step for malaria transmission, using insecticides to control the mosquito population has traditionally been an effective method to prevent the disease. However, the spread of insecticide resistance in mosquito populations and the lack of vaccines against the disease have prompted the public health community to advocate new strategies for malaria control.

During malaria transmission from a host to mosquitoes, some mosquito proteins, such as Tep1, APL1C, and LRIM1, inhibit Plasmodium infection of mosquitoes, while other mosquito proteins, such as the fibrinogen-related protein 1 (FREP1) that binds to parasites in the mosquito midgut, facilitate Plasmodium invasion. Antibodies against FREP1 inhibit infection by P. vivax, P. falciparum, and P. berghei of Anopheles dirus and An. gambiae mosquitoes, supporting the hypothesis that this pathway is conserved across multiple Plasmodium and Anopheles species.

FREP1 belongs to the fibrinogen-related protein family whose members contain a conserved fibrinogen-like domain FBG with approximately 200 amino acids. In mammals, fibrinogens are involved in blood coagulation, whereas in invertebrates, they function as pattern recognition receptors capable of binding to bacteria, fungi, or parasites. Since mosquito FREP1 facilitates Plasmodium infection through direct binding to gametocytes and ookinetes, small molecules that interrupt this interaction can be ideal candidates to block malaria transmission. Such compounds can be administered to malaria patients or be sprayed outdoors, indoors, or on bed nets. At present, very few preparations are available in the market for this purpose.

Thus, there is a need for identifying and developing small molecules that can interrupt malaria transmission and further can treat and prevent malaria infection. In particular, there is a need for identifying and developing transmission-blocking agents or drugs that inhibit malaria transmission, for example, via FREP1-mediated malaria transmission pathway.

### BRIEF SUMMARY OF THE INVENTION

The subject invention provides fungal extracts, fungal metabolites, pharmaceutical compositions comprising the fungal extracts, and/or fungal metabolites, methods of preparation, and therapeutic uses thereof. Advantageously, the subject fungal extracts, fungal metabolites, and pharmaceutical compositions comprising the fungal extracts, and/or fungal metabolites, can be used to treat, inhibit and/or prevent malaria infection and transmission.

In a preferred embodiment, the fungal strain is a Purpureocillium species. In a specifically, the fungal strain is Purpureocillium lilacinum.

In one embodiment, the fungal extract comprises one or more fungal metabolites.

The subject invention also provides fungal metabolites isolated from the subject fungal extracts, therapeutic or pharmaceutical compositions comprising a therapeutically effective amount of the subject fungal metabolites and, optionally, a pharmaceutically acceptable carrier.

In one embodiment, the fungal extracts/metabolites, and/or the composition comprising the fungal extracts/metabolites comprises a bioactive agent isolated from the fungal strain, e.g., Purpureocillium lilacinum. Preferably, the bioactive agent is pulixin that can stop malaria transmission to mosquitoes, inhibiting malaria infection and inhibiting parasite proliferation.

In one embodiment, the subject invention provides a fungal extract spray or aerosol comprising the fungal extracts, fungal metabolites or compositions comprising the fungal extracts and/or fungal metabolites. The fungal extract/metabolite sprays can protect humans from malaria infection.

In one embodiment, the subject invention provides an antimalaria agent/compound having a general structure of formula (I):

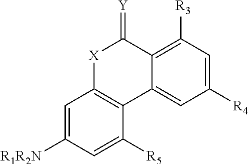

wherein X and Y are independently selected from S, N and O; R₁ and R₂ are independently selected from hydrogen, alkyl and substituted alkyl; and R₃, R₄ and R₅ are independently selected from hydrogen, alkyl, substituted alkyl, —NR₁R₂, and —OR₆, wherein R₆ is hydrogen, alkyl, aryl, substituted alkyl or substituted aryl.

In one embodiment, the antimalaria agent/compound and compositions comprising the antimalaria agent/compound can be used to inhibit malaria infection and transmission to mosquitos.

The subject invention provides methods of treating, inhibiting or preventing malaria infection in a subject in need thereof comprising administering the fungal extracts, the fungal metabolites or the composition of the subject invention to the subject in an amount effective to treat, inhibit, or prevent malaria infection in the subject

Also provided are methods of inhibiting malaria transmission to mosquitoes, the methods comprising exposing mosquitoes to the fungal extracts, the fungal metabolites or the composition of the subject invention in an amount effective to inhibit malaria transmission.

In one embodiment, the subject invention provides a method of inhibiting the FREP1-parasite interaction.

### BRIEF DESCRIPTION OF THE DRAWINGS

FIGS. 1A-1B show the candidate fungal extract blocks P. falciparum transmission through feeding or spraying. (a) The fungal extract (GFEL-12E6) significantly inhibited P. falciparum infection in mosquito midguts in the SMFA. The experiment was independently repeated three times and the results were consistent. (b) Exposure to the fungal extract significantly inhibited P. falciparum infection in mosquito midguts. The fungal extract in acetone was sprayed on to cups and dried. Mosquitoes were placed in the cup for 24 hours before the SMFA. This showed one experiment and the experiment was independently repeated three times, and the results were consistent. The gray line indicates the median number of oocysts in mosquitoes in each treatment. The bold lines at zero oocysts indicate many mosquitoes with zero oocysts. N: the number of mosquitoes in the group; Infection (%): the percentage of infected mosquitoes; and Median: the median number of oocysts in mosquito midguts.

FIGS. 2A-2I show the candidate fungus identified as Purpureocillium lilacinum. Colonies for observation were grown on potato dextrose agar (PDA) medium plate for 7-15 days at 25° C. (a) Short conidiophores; (b) long conidiophores; (c) solitary phialide-producing catenate conidia; Arrows in (a, b, c) point to phialides. (d, e) typical sub-globose conidia; (f) cylindrical conidia; (g) colony surface on PDA medium plate (top); (h) colony reverse on PDA (bottom). scale bar: 10 μM. (i) Maximum Parsimony tree was constructed based on ITS sequences and bootstrap values above 50% are indicated at the nodes.

FIGS. 3A-3D show the spectrum for the isolated pure compound from GFEL-12E6. (a) The HPLC profile of the pure compound shows one peak. (b) The absorbance spectrum of the purified compound. (c) The crystal of pulixin. (d) The structure of pulixin. Since DMSO was used as a solvent to grow a crystal, DMSO formed a hydrogen bond with pulixin.

FIG. 4 shows the mass spectrum of pulixin. The identification of the candidate compound through the mass spectrometry profile of pulixin, showing a mass of 258.0764, matched the calculated mass.

FIG. 5 shows the ¹H-NMR spectrum of pulixin. The ¹H-NMR profile of pulixin confirmed the proposed structure.

FIG. 6 shows the ¹³C-NMR spectrum of pulixin. The ¹³C-NMR profile was consistent with the proposed structure.

FIGS. 7A-7D show that Pulixin inhibits the FREP1-P. falciparum interaction and blocks malaria transmission. (a) ELISA results showed that pulixin inhibited the interaction between FREP1 and P. falciparum-infected cell lysate and the inhibition was dose-dependent. P: The positive control by using the heat-inactivated FREP1 that did not interact with parasites. (b) The midguts of pulixin treated mosquitoes had fewer oocysts than those of the control (DMSO) mosquitoes. Dots inside the midguts are oocysts. (c) Pulixin inhibited the transmission of P. falciparum to An. gambiae in a dose-dependent manner. This experiment was independently conducted twice, and the results were similar. Each dot represents the number of oocysts in an experimental mosquito. Gray lines show the median number of oocysts. N: number of mosquitoes. Median: median number of oocysts. Infection (%): Percentage of infected mosquitoes. (d) Pulixin did not inhibit the formation of ookinetes. The assay was independently conducted twice. Each repeat had three replicates in the experimental and control groups. The conversion rate was defined as (number of ookinetes/number of gametocytes)×100%. Bold lines depict the means of conversion rates. Con: control groups with DMSO in culture; Exp: experimental groups with 40 μM of pulixin in culture.

FIGS. 8A-8B show that Pulixin was able to inhibit the development of the asexual-stage P. falciparum in blood. The test for each concentration was replicated three times and the assays were repeated. The profiles show the means and standard errors. (a) Parasitemia at day 1, 2, 3, and 4 after inoculation with P. falciparum-infected blood without pulixin. Significant more (p<0.001) parasite-infected cells at day 4 comparing to day 1. (b) Parasitemia on day 4 after incubated with different concentrations of pulixin. Significantly fewer P. falciparum-infected cells were observed when the concentration of pulixin was greater than 0.01 μg/mL (p<0.05), compared to the control (0 μg/mL pulixin).

FIGS. 9A-9B shows that Pulixin did not show significant cytotoxicity to the human embryonic kidney 293 cell line at a concentration of 30 μg/mL or lower. (a) The cytotoxic effects of pulixin on human embryonic kidney 293 (HEK293) cell proliferation at varying concentrations (0-100 μg/mL) were measured with MTT assays. No significant difference (p=0.89) was observed when the concentration of pulixin was 30 μg/mL or lower. The density of living cells was significantly lower when pulixin reached 100 μg/mL, compared to the other concentrations (p<0.03). The test for each concentration was replicated three times. The data were analyzed using ANOVA. (b) Cells were observed under bright-field microscopy. Consistent with MTT assays, much fewer cells were observed when the concentration of pulixin reached 100 μg/mL than under other concentrations, e.g., pulixin ≤30 μg/mL.

### BRIEF DESCRIPTION OF SEQUENCES

SEQ ID NO: 1 is the sequence of a ITS1F primer for the nuclear ribosomal internal transcribed spacer (ITS) region contemplated for use according to the subject invention.

SEQ ID NO: 2 is the sequence of a ITS4 primer for the ITS region contemplated for use according to the subject invention.

### DETAILED DESCRIPTION OF THE INVENTION

The subject invention provides fungal extracts, fungal metabolites, pharmaceutical compositions comprising the fungal extracts, and/or fungal metabolites, methods of preparation, and therapeutic uses thereof. Advantageously, the subject fungal extracts, fungal metabolites, and pharmaceutical compositions comprising the fungal extracts, and/or fungal metabolites, can be used to treat, inhibit and/or prevent malaria infection and transmission.

The subject invention provides efficient and convenient methods for preparing fungal extracts. In one embodiment, the fungal extract is prepared at room temperature, using, for example, ethanol, methanol, ethyl acetate, acetone, acetyl acetate and any combination thereof, as the solvent.

In one embodiment, the method for preparing a fungal extract comprises the steps of: a) culturing the fungus or providing a sufficient quantity of fungal culture; b) extracting the fungal culture with a solvent at, for example, room temperature to yield an extract; and c) recovering the extract. In a preferred embodiment, the solvent is selected from hexane, dichloromethane, ethanol, methanol, ethyl acetate, acetone, acetyl acetate and any combination thereof.

In one embodiment, the method for isolating a fungal metabolite comprises the steps of: a) culturing the fungus or providing a sufficient quantity of fungal culture; b) extracting the fungal culture with a solvent at, for example, room temperature to yield an extract; c) recovering the extract; and d) isolate the fungal metabolite with a second solvent at, for example, room temperature.

In a further embodiment, the second solvent can be the same as or different from the first solvent. The second solvent is selected from, for example, hexane, dichloromethane, ethanol, methanol, ethyl acetate, acetone, acetyl acetate and any combination thereof. In another embodiment, the fungal extract can be obtained via sequential extraction, by extracting the solvent-extract with a different solvent each time to extract the desired fungal metabolite.

In one embodiment, the fungal strains of the subject invention are isolated from soil, water, air, other organisms, or plants. In a preferred embodiment, the fungal strain is a Purpureocillium species. In a specifically, the fungal strain is Purpureocillium lilacinum.

The subject invention provides fungal extracts produced by the subject extraction methods. Also provided are therapeutic or pharmaceutical compositions comprising a therapeutically effective amount of the subject fungal extract, and, optionally, a pharmaceutically acceptable carrier.

An extract is a concentrated preparation of the essential constituents of a raw material, e.g., fungus. Typically, the essential constituents are extracted from the raw materials by suspending the raw materials in an appropriate choice of solvent. The extracting process may be further facilitated by means of, for example, maceration, percolation, repercolation, counter-current extraction, turbo-extraction, or by carbon-dioxide hypercritical (temperature/pressure) extraction. After filtration to rid of debris, the extracting solution may be further evaporated and thus concentrated to yield a soft extract and/or eventually a dried extract by means of, for example, spray drying, vacuum oven drying, fluid-bed drying or freeze-drying. The soft extract or dried extract may be further dissolved in a suitable liquid to a desired concentration for administering or processed into a form such as pills, capsules, injections, etc.

In one embodiment, the fungal extract may be a crude extract. In one embodiment, the fungal extract can be further evaporated to produce solid or semi-solid compositions. In another embodiment, the fungal extract may be concentrated and/or purified. In one embodiment, the solid or semi-solid fungal extract may be dissolved in a third solvent, e.g., DMSO, to produce a liquid composition. In one embodiment, the third solvent may be the same or different from the first and second solvent.

In certain embodiments, suitable solvents for the preparation of fungal extract/metabolite include, but are not limited to, alcohols (e.g., methanol, ethanol, propanol, and butanol); ketones (e.g., acetone) or alkyl ketones; chloroform; acetic acid; butyl acetate, dimethyl sulfoxide, ethyl acetate, ethyl ether, ethyl formate, formic acid, heptane, isobutyl acetate, isopropyl acetate, and methyl acetate.

In one embodiment, the fungal culture is mixed with the solvent for at least about 30 minutes to produce a fungal extract. Preferably, the extraction time is at least about 40 minutes, 50 minutes, 1 hour, 1.5 hours, 2 hours, 2.5 hours, 3 hours, 4 hours, 5 hours, 6 hours, 7 hours, 8 hours, 9 hours, or 10 hours.

In one embodiment, the fungal extract comprises one or more fungal metabolites. In one embodiment, the fungal extract comprises one or more molecules secreted from one or more fungi.

The subject invention also provides fungal metabolites isolated from the subject fungal extracts, therapeutic or pharmaceutical compositions comprising a therapeutically effective amount of the subject fungal metabolites and, optionally, a pharmaceutically acceptable carrier.

“Pharmaceutically acceptable carrier” refers to a diluent, adjuvant or excipient with which the antigen disclosed herein can be formulated. Typically, a “pharmaceutically acceptable carrier” is a substance that is non-toxic, biologically tolerable, and otherwise biologically suitable for administration to a subject, such as an inert substance, added to a pharmacological composition or otherwise used as a diluent, or excipient to facilitate administration of the antigen disclosed herein and that is compatible therewith. Examples of excipients include various sugars and types of starches, cellulose derivatives, gelatin, vegetable oils, and polyethylene glycols. Additional examples of carriers suitable for use in the pharmaceutical compositions are known in the art and such embodiments are within the purview of the invention.

Examples of carriers suitable for use in the pharmaceutical compositions are known in the art and such embodiments are within the purview of the invention. The pharmaceutically acceptable carriers and excipients, including, but not limited to, aqueous vehicles, water-miscible vehicles, non-aqueous vehicles, stabilizers, solubility enhancers, isotonic agents, buffering agents, suspending and dispersing agents, wetting or emulsifying agents, complexing agents, sequestering or chelating agents, cryoprotectants, lyoprotectants, thickening agents, pH adjusting agents, and inert gases. Other suitable excipients or carriers include, but are not limited to, dextran, glucose, maltose, sorbitol, xylitol, fructose, sucrose, and trehalose.

In one embodiment, the composition of the subject invention comprises a fungal extract and, optionally, a pharmaceutically acceptable carrier. Preferably, the fungus being a strain from Purpureocillium species, such as Purpureocillium lilacinum. In one embodiment, the fungal extract comprises one or more bioactive fungal metabolites. In one embodiment, the fungal extract is a hexane, dichloromethane, ethanol, methanol, ethyl acetate, acetone, or acetyl acetate extract.

In certain embodiments, the composition of the subject invention further comprises an extract from the fungal strain Penicillium thomii, Penicillium pancosmium, Aspergillus niger, and/or Aspergillus aculeatus. In some embodiments, the composition of the subject invention comprises two or more fungal extracts from the fungal strains Purpureocillium lilacinum, Penicillium thomii, Penicillium pancosmium, Aspergillus niger, and Aspergillus aculeatus.

In certain embodiments, the composition of the subject invention further comprises a fungal metabolite isolated from the fungal extracts of Penicillium thomii, Penicillium pancosmium, Aspergillus niger, and/or Aspergillus aculeatus. In a specific embodiment, the composition of the subject invention further comprises one or more fungal metabolites selected from asperaculane B, and P-orlandin.

In one embodiment, the subject invention provides a method comprising creating a chemical profile for the fungal extract, by using, for example, high performance liquid chromatography (HPLC), gas chromatography-mass spectrometry (GC-MS), NMR and/or crystallography.

In one embodiment, the isolated fungal metabolite has a general structure of formula (I):

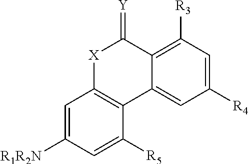

wherein X and Y are independently selected from S, N and O; R₁ and R₂ are independently selected from hydrogen, alkyl and substituted alkyl; and R₃, R₄ and R₅ are independently selected from hydrogen, alkyl, substituted alkyl, —NR₁R₂, and —OR₆, wherein R₆ is hydrogen, alkyl, aryl, substituted alkyl or substituted aryl.

As used herein, “alkyl” means saturated monovalent radicals of at least one carbon atom or a branched saturated monovalent of at least three carbon atoms, e.g., C₁-C₁₀ alkyl. It may include straight-chain alkyl groups, branched-chain alkyl groups, cycloalkyl (alicyclic) groups, alkyl substituted cycloalkyl groups, and cycloalkyl substituted alkyl groups. It may include hydrocarbon radicals of at least one carbon atom, which may be linear. Examples include, but are not limited to, methyl, ethyl, propyl, 2-propyl, n-butyl, iso-butyl, tert-butyl, pentyl, hexyl, and the like.

As used herein, “aryl” refers to a carbocyclic (all carbon) monocyclic or multicyclic aromatic ring system (including fused ring systems where two carbocyclic rings share a chemical bond). The number of carbon atoms in an aryl group can vary. For example, the aryl group can be a C₆-C₁₄ aryl group, a C₆-C₁₀ aryl group, or a C₆ aryl group. Examples of aryl groups include, but are not limited to, phenyl, benzyl, α-naphthyl, β-naphthyl, biphenyl, anthryl, tetrahydronaphthyl, fluorenyl, indanyl, biphenylenyl, and acenaphthenyl. Preferred aryl groups are phenyl and naphthyl.

As used herein, a “substituted” group may be substituted with one or more group(s) individually and independently selected from alkyl, alkenyl, benzyl, aryl, hydroxyl, alkoxy, acyl, halogen, thiol, and amino.

In a specific embodiment, the isolated fungal metabolite is pulixin, 3-amino-7,9-dihydroxy-1-methyl-6H-benzo[c]chromen-6-one, having the chemical structure of

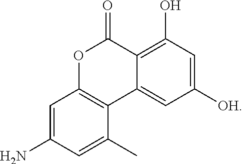 [SMILES: Cc1cc(N)cc2oc(=O)c3c(O)cc(O)cc3c12]

The crystal data have been submitted to the Cambridge Crystallographic Data Centre with the deposition number 2005130.

Pulixin prevented FREP1 from binding to P. falciparum-infected cell lysate. Pulixin blocked the transmission of the parasite to mosquitoes with the EC₅₀ of 11 μM based on SMFA. Notably, pulixin also inhibited the proliferation of the asexual-stage P. falciparum with the EC₅₀ of 47 nM.

In one embodiment, the fungal extracts/metabolites, and/or the composition comprising the fungal extracts/metabolites comprises a bioactive agent pulixin that is capable of stopping malaria transmission to mosquitoes, inhibiting malaria infection and inhibiting parasite proliferation.

In one embodiment, the fungal extracts, fungal metabolites and compositions comprising the fungal extracts and/or fungal metabolites blocks the interaction between FREP1 protein from the midgut of a mosquito and a malaria parasite, which in turn, inhibits the malaria transmission.

In one embodiment, the fungal extracts, fungal metabolites, the bioactive agent, and compositions of the subject invention can be administered to the subject being treated by standard routes, including oral, inhalation, or parenteral administration including intravenous, subcutaneous, topical, transdermal, intradermal, transmucosal, intraperitoneal, intramuscular, intracapsular, intraorbital, intracardiac, transtracheal, subcutaneous, subcuticular, intraarticular, subcapsular, subarachnoid, intraspinal, epidural and intrasternal injection, infusion, and electroporation.

In one embodiment, the composition is formulated in accordance with routine procedures as a pharmaceutical composition adapted for local administration to a subject, e.g., humans. Typically, compositions for local administration are solutions in a sterile isotonic aqueous buffer. Generally, the ingredients are supplied either separately or mixed together in unit dosage form, for example, as a dry lyophilized powder or water free concentrate in a hermetically sealed container such as an ampoule or sachette indicating the quantity of active agent.

In one embodiment, the fungal extracts, fungal metabolites and the pharmaceutical composition of the subject invention may be formulated in the forms of powders, dressings, creams, ointments, solutions, micellar solutions, emulsions, microemulsions, pastes, suspensions, gels, foams, oils, aerosols, granules, solids, or sprays. Preferrably, the fungal extracts, fungal metabolites and compositions comprising the fungal extracts and/or fungal metabolites can be formulated in a spray or aerosol. The fungal extract/metabolite sprays or aerosol can protect humans from malaria infection.

In one embodiment, the fungal extract may be formulated in a container as a fungal extract spray. The fungal extract spray can be applied onto any surface a mosquito may be sitting or landing, for example, human skin, wall surface, floor surface, and a surface of an object, such as furniture.

In one embodiment, the subject invention provides a fungal spray comprising a fungal extract and/or a fungal metabolite. Preferably, the fungal extract is produced from a Purpureocillium species, e.g., Purpureocillium lilacinum. In certain embodiments, the fungal spray further comprises an extract from the fungal strain Penicillium thomii, Penicillium pancosmium, Aspergillus niger, and/or Aspergillus aculeatus.

In a specific embodiment, the fungal spray comprises a fungal metabolite having a general structure of formula (I):

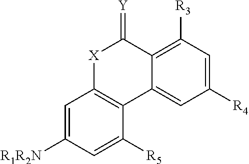

wherein X and Y are independently selected from S, N and O; R₁ and R₂ are independently selected from hydrogen, alkyl and substituted alkyl; and R₃, R₄ and R₅ are independently selected from hydrogen, alkyl, substituted alkyl, —NR₁R₂, and —OR₆, wherein R₆ is hydrogen, alkyl, aryl, substituted alkyl or substituted aryl.

In a specific embodiment, the fungal metabolite is pulixin. In specific embodiments, the spray further comprises one or more fungal metabolites selected from asperaculane B and P-orlandin.

In some embodiments, the fungal spray also comprise an insecticide selected from organochlorides, organophosphates, carbamates, pyrethroids, neonicotinoids, butenolides, ryanoids and diamides.

In one embodiment, the fungal spray may further comprise a cytochrome b inhibitor such as atovaquone.

In one embodiment, the fungal spray may be applied onto the surfaces with fungal metabolites at, for example, at least about 0.1 μg/cm², about 0.2 μg/cm², about 0.5 μg/cm², about 1 μg/cm², about 1.5 μg/cm², about 2 μg/cm², about 2.5 μg/cm², about 3 μg/cm², about 3.5 μg/cm², about 4 μg/cm², about 4.5 μg/cm², about 5 μg/cm², about 5.5 μg/cm², about 6 μg/cm², about 6.5 μg/cm², about 7 μg/cm², about 7.5 μg/cm², about 8 μg/cm², about 8.5 μg/cm², about 9 μg/cm², about 10 μg/cm², or any amount in between.

In one embodiment, the fungal spray may be applied onto the surfaces with fungal metabolites, for example, from about 1 mg/m² to about 100 mg/m², from about 1 mg/m² to about 90 mg/m², from about 1 mg/m² to about 80 mg/m², from about 1 mg/m² to about 70 mg/m², from about 1 mg/m² to about 60 mg/m², from about 1 mg/m² to about 50 mg/m², from about 2 mg/m² to about 50 mg/m², from about 5 mg/m² to about 50 mg/m², from about 10 mg/m² to about 50 mg/m², from about 10 mg/m² to about 40 mg/m², or from about 20 mg/m² to about 40 mg/m².

In one embodiment, the fungal spray maybe applied at least every hour, every two hours, every three hours, every four hours, every five hours, every six hours, every seven hours, every eight hours, every nine hours, every ten hours, every eleven hours, every twelve hours, or once a day.

The subject invention further provides methods of treating, inhibiting, or preventing malaria infection in a subject in need thereof comprising administering the fungal extracts, the fungal metabolites or the composition of the subject invention to the subject in an amount effective to treat, inhibit, or prevent malaria infection in the subject.

The term “amount effective,” as used herein, refers to an amount that is capable of treating or ameliorating a disease or condition or otherwise capable of producing an intended therapeutic effect. In certain embodiments, the effective amount enables at least 5%, 10%, 15%, 20%, 25%, 30%, 35%, 40%, 45%, 50%, 60%, 70%, 80%, 90%, or 100% inhibition of malaria infection and transmission.

In one embodiment, the administration to a subject can be via any convenient and effective route, such oral, rectal, nasal, topical, (including buccal and sublingual), transdermal, parenteral (including intramuscular, subcutaneous, and intravenous), spinal (epidural, intrathecal), and central (intracerebroventricular). Non-limiting embodiments include parenteral administration, such as by injection, e.g., into the blood stream, intradermal, intramuscular, etc., or mucosal administration, e.g., intranasal, oral, and the like.

In certain embodiments, the malaria parasite is selected from P. falciparum, P. malariae, P. ovale, P. vivax, P. knowlesi, P. berghei, P. chabaudi and P. yoelii. In specific embodiments, malaria is caused by, for example, Plasmodium (P.) species including P. falciparum, P. malariae, P. ovale, P. vivax, P. knowlesi, P. berghei, P. chabaudi and P. yoelii.

In one embodiment, the subject may be any animal including mammals, preferably, human. The subjects further include, but are not limited to, non-human primates, rodents (e.g., rats, mice), dogs, cats, horses, cattle, pigs, sheep, goats, chickens, guinea pigs, hamsters and the like.

The term “prevention” or any grammatical variation thereof (e.g., prevent, preventing, etc.), as used herein, includes but is not limited to, at least the reduction of likelihood of the risk of (or susceptibility to) acquiring a disease or disorder (i.e., causing at least one of the clinical symptoms of the disease not to develop in a patient that may be exposed to or predisposed to the disease but does not yet experience or display symptoms of the disease). The term “prevention” may refer to avoiding, delaying, forestalling, or minimizing one or more unwanted features associated with a disease or disorder, and/or completely or almost completely preventing the development of a disease or disorder and its symptoms altogether. Prevention can further include, but does not require, absolute or complete prevention, meaning the disease or disorder may still develop at a later time and/or with a lesser severity than it would without preventative measures. Prevention can include reducing the severity of the onset of a disease or disorder, and/or inhibiting the progression thereof.

The terms “treatment” or any grammatical variation thereof (e.g., treat, treating, etc.), as used herein, includes but is not limited to, the application or administration to a subject (or application or administration to a cell or tissue from a subject) with the purpose of delaying, slowing, stabilizing, curing, healing, alleviating, relieving, altering, remedying, less worsening, ameliorating, improving, or affecting the disease or condition, the symptom of the disease or condition, or the risk of (or susceptibility to) the disease or condition. The term “treating” refers to any indication of success in the treatment or amelioration of a pathology or condition, including any objective or subjective parameter such as abatement; remission; lessening of the rate of worsening; lessening severity of the disease; stabilization, diminishing of symptoms or making the pathology or condition more tolerable to the subject; or improving a subject's physical or mental well-being.

In one embodiment, the method of treating, inhibiting or preventing malaria infection in a subject comprises administering a pharmaceutical composition comprising a fungal extract to the subject, wherein the fungal extract is a Purpureocillium lilacinum extract. Preferably, the Purpureocillium lilacinum extract comprises a bioactive fungal metabolite having a general structure of formula (I):

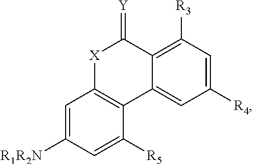

wherein X and Y are independently selected from S, N and O; R₁ and R₂ are independently selected from hydrogen, alkyl and substituted alkyl; and R₃, R₄ and R₅ are independently selected from hydrogen, alkyl, substituted alkyl, —NR₁R₂, and —OR₆, wherein R₆ is hydrogen, alkyl, aryl, substituted alkyl or substituted aryl.

In a specific embodiment, the method of treating, inhibiting or preventing malaria infection in a subject comprises administering a pharmaceutical composition comprising a fungal extract to the subject, wherein the fungal extract comprises pulixin.

In one embodiment, the method of treating, inhibiting or preventing malaria infection in a subject comprises administering a pharmaceutical composition comprising a bioactive fungal metabolite from Purpureocillium lilacinum, the bioactive fungal metabolite having a general structure of formula (I):

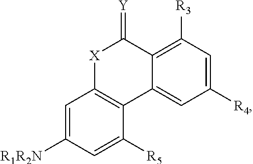

wherein X and Y are independently selected from S, N and O; R₁ and R₂ are independently selected from hydrogen, alkyl and substituted alkyl; and R₃, R₄ and R₅ are independently selected from hydrogen, alkyl, substituted alkyl, —NR₁R₂, and —OR₆, wherein R₆ is hydrogen, alkyl, aryl, substituted alkyl or substituted aryl.

In a specific embodiment, the method of treating, inhibiting or preventing malaria infection in a subject comprises administering a pharmaceutical composition comprising pulixin.

In one embodiment, a suitable dose will be in the range of from about 0.001 to about 100 mg/kg of body weight per day, preferably from about 0.01 to about 100 mg/kg of body weight per day, more preferably, from about 0.1 to about 50 mg/kg of body weight per day, or even more preferred, in a range of from about 1 to about 10 mg/kg of body weight per day. For example, a suitable dose may be about 1 mg/kg, 10 mg/kg, or 50 mg/kg of body weight per day.

The fungal extracts, fungal metabolites can be administered to achieve peak plasma concentrations of, for example, from about 0.005 to about 200 μM, from about 0.01 to about 150 μM, from about 0.02 to about 100 μM, from about 0.02 to about 80 μM, from about 0.05 to about 50 μM, from about 0.05 to about 20 μM, from about 0.05 to about 10 μM, from about 0.05 to about 5 μM, from about 0.05 to about 1 μM, from about 0.1 to about 100 μM, from about 0.5 to about 75 μM, from about 1 to about 50 μM, from about 2 to about 30 μM, or from about 5 to about 25 μM.

Also provides are methods of inhibiting/reducing/preventing malaria transmission to mosquitoes, the methods comprising exposing mosquitoes to the fungal extracts, the fungal metabolites or the composition of the subject invention in an amount effective to inhibit/reduce/prevent malaria transmission. Said exposing comprising contacting/feeding mosquitos with the fungal extracts, fungal metabolites or the composition of the subject invention.

Examples of mosquito genera include, but are not limited to Aedeomyia, Aedes, Anopheles, Armigeres, Ayurakitia, Borachinda, Coquillettidia, Culex, Culiseta, Deinocerites, Eretmapodites, Ficalbia, Galindomyia, Haemagogus, Heizmannia, Hodgesia, Isostomyia, Johnbelkinia, Kimia, Limatus, Lutzia, Malaya, Mansonia, Maorigoeldia, Mimomyia, Onirion, Opifex, Orthopodomyia, Psorophora, Runchomyia, Sabethes, Shannoniana, Topomyia, Toxorhynchites, Trichoprosopon, Tripteroides, Udaya, Uranotaenia, Verrallina, and Wyeomyia. In one embodiment, the mosquito is an Anopheles spp., Aedes spp., Culex spp., Culiseta spp., Haemagogus spp. Preferably, the mosquito may be Anopheles spp.

In a further embodiment, the Anopheles spp. may be An. arabiensis, An. funestus, An. gambiae, An. moucheti, An. nili, An. stephensi, An. bellator, An. cruzii, An. farauti or a combination of two or more thereof. Preferably, the Anopheles spp. may be An. gambiae. Examples of the Anopheles species include Anopheles (Cellia) aconitus; Anopheles (Nyssorhynchus) albimanus; Anopheles (Nyssorhynchus) albitarsis species complex; Anopheles (Cellia) annularis; Anopheles (Nyssorhynchus) aquasalis; Anopheles (Cellia) arabiensis; Anopheles (Anopheles) atroparvus; Anopheles (Cellia) balabacensis; Anopheles (Anopheles) barbirostris species complex; Anopheles (Cellia) culicifacies species complex; Anopheles (Nyssorhynchus) darling; Anopheles (Cellia) dirus species complex; Anopheles (Cellia) farauti species complex; Anopheles (Cellia) flavirostris; Anopheles (Cellia) fluviatilis species complex; Anopheles (Anopheles) freehorni; Anopheles (Cellia) funestus; Anopheles (Cellia) gambiae; Anopheles (Cellia) koliensis; Anopheles (Anopheles) labranchiae; Anopheles (Anopheles) lesteri (formerly An. anthropophagus in China); Anopheles (Cellia) leucosphyrus and Anopheles (Cellia) latens; Anopheles (Cellia) maculatus Group; Anopheles (Nyssorhynchus) marajoara; Anopheles (Cellia) melas; Anopheles (Cellia) merus; Anopheles (Anopheles) messeae; Anopheles (Cellia) minimus species complex; Anopheles (Cellia) moucheti; Anopheles (Cellia) nili species complex; Anopheles (Nyssorhynchus) nuneztovari species complex; Anopheles (Anopheles) pseudopunctipennis species complex; Anopheles (Cellia) punctulatus species complex; Anopheles (Anopheles) quadrimaculatus; Anopheles (Anopheles) sacharovi; Anopheles (Cellia) sergentii species complex; Anopheles (Anopheles) sinensis species complex; Anopheles (Cellia) stephensi; Anopheles (Cellia) subpictus species complex; Anopheles (Cellia) sundaicus species complex; Anopheles (Cellia) superpictus.

In an embodiment, the mosquito is female.

In one embodiment, the method of inhibiting/reducing/preventing malaria transmission comprises exposing a mosquito to a composition comprising a fungal extract, wherein said exposing comprising contacting/feeding/spraying the mosquito with the composition comprising the fungal extract, wherein the fungal extract is a Purpureocillium lilacinum extract. Preferably, the Purpureocillium lilacinum extract comprises a bioactive fungal metabolite having a general structure of formula (I):

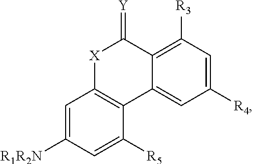

wherein X and Y are independently selected from S, N and O; R₁ and R₂ are independently selected from hydrogen, alkyl and substituted alkyl; and R₃, R₄ and R₅ are independently selected from hydrogen, alkyl, substituted alkyl, —NR₁R₂, and —OR₆, wherein R₆ is hydrogen, alkyl, aryl, substituted alkyl or substituted aryl.

In one embodiment, the method of inhibiting/reducing/preventing malaria transmission comprises exposing a mosquito to a pharmaceutical composition comprising a bioactive fungal metabolite from Purpureocillium lilacinum, the bioactive fungal metabolite having a general structure of formula (I):

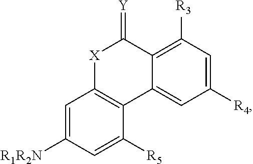

wherein X and Y are independently selected from S, N and O; R₁ and R₂ are independently selected from hydrogen, alkyl and substituted alkyl; and R₃, R₄ and R₅ are independently selected from hydrogen, alkyl, substituted alkyl, —NR₁R₂, and —OR₆, wherein R₆ is hydrogen, alkyl, aryl, substituted alkyl or substituted aryl.

In a specific embodiment, the method of inhibiting/reducing/preventing malaria transmission comprises exposing a mosquito to a composition comprising pulixin, wherein said exposing comprises contacting/feeding/spraying the mosquito with the composition comprising pulixin.

In one embodiment, the method of inhibiting/reducing/preventing malaria transmission may comprise spraying a surface a mosquito may be sitting or landing with the fungal spray of the subject invention. Preferably, the method of inhibiting/reducing/preventing malaria transmission comprises spraying a human with the fungal spray of the subject invention. In a specific embodiment, the human has been diagnosed with malaria or is suffering from malaria.

In one embodiment, the subject invention provides a method of inhibiting/reducing/preventing the interaction of malaria parasite and a mosquito, the method comprising exposing the mosquito to a fungal extract, a fungal metabolite or a pharmaceutical composition comprising the fungal extract, or fungal metabolite of the subject invention, wherein said exposing comprises contacting/feeding/spraying the mosquito with the fungal extract, the fungal metabolite or the pharmaceutical composition comprising the fungal extract, or the fungal metabolite of the subject invention.

In one embodiment, the method of inhibiting/reducing/preventing the interaction of malaria parasite and a mosquito comprises spraying a human with the fungal spray of the subject invention. In a specific embodiment, the human has been diagnosed with malaria or is suffering from malaria.

The subject invention also provides methods for treating a subject infected with malaria parasites or malaria parasite oocysts. The subject invention further provides methods for treating a subject suffering from malaria. Further provided in the subject invention are methods of preventing or reducing malaria transmission from a subject infected with a malarial parasite, comprising administering to the subject a fungal extract/metabolite or the composition of the subject invention that blocks the interaction between malarial parasite and FREP-1 from the midgut of a mosquito.

In one embodiment, the subject invention provides a method of inhibiting the FREP1-parasite interaction, the method comprising exposing or feed the mosquito with the fungal extracts/metabolite or composition of the subject invention.

In one embodiment, the subject invention provides a method of inhibiting the interaction between the midgut proteins, for example, a mosquito FREP1 protein, and parasite surface antigens, for example, Hsp 70, and α-tubulin 1.

In some embodiments, the infection of a malaria parasite in the mosquito may be interrupted by blocking the invasion of the malaria parasite into the midgut of the mosquito; inhibiting the penetration of the malaria parasite through the midgut peritrophic matrix (PM); and/or blocking the recognition between the malaria parasite and the midgut PM.

In one embodiment, the subject invention provides a method of inhibiting/reducing/preventing the malaria infection in a mosquito, the method comprising exposing the mosquito to the fungal extract, fungal metabolite or composition comprising the fungal extract or fungal metabolite of the subject invention, wherein said exposing comprises spraying a surface where the mosquito may sit or land with the fungal extract, fungal metabolite or composition comprising the fungal extract, or fungal metabolite of the subject invention, and/or contacting/feeding/spraying the mosquito with the fungal extract, fungal metabolite or pharmaceutical composition comprising the fungal extract, or fungal metabolite of the subject invention.

In a specific embodiment, the method of inhibiting or preventing malaria infection in a mosquito comprises exposing the mosquito to the fungal extract comprising pulixin, or the composition comprising pulixin, wherein said exposing comprises spraying a surface with the fungal extract comprising pulixin, or the composition comprising pulixin, and/or contacting/feeding/spraying the mosquito with the fungal extract comprising pulixin, or the composition comprising pulixin.

In one embodiment, the subject invention provides a method of inhibiting/reducing/preventing the malaria infection in a mosquito, the method comprising administering to the mosquito the fungal extract, fungal metabolite or composition comprising the fungal extract or fungal metabolite of the subject invention. In a preferred embodiment, administering to the mosquito comprises feeding the mosquito a sample comprising the fungal extract, fungal metabolite or composition comprising the fungal extract or fungal metabolite of the subject invention. In a specific embodiment, the sample is a blood sample of a subject, e.g., human, having been administered with the fungal extract, the fungal metabolite or the composition comprising the fungal extract or fungal metabolite of the subject invention. In certain embodiments, the blood sample comprises malaria parasites.

In one embodiment, the subject invention provides a method for inhibiting, or reducing the amount of malaria oocytes in the mosquito, the method comprising exposing the mosquito/administering to the mosquito a fungal extract/metabolite or a composition of the subject invention. Preferably, said exposing comprises spraying a surface where a mosquito may sit or land with the fungal spray of the subject invention.

In one embodiment, the subject invention provides a method for increasing the resistance of a mosquito to malaria parasite, the methods comprising exposing mosquitoes to the fungal extracts, the fungal metabolites or the composition of the subject invention. Preferably, said exposing comprises spraying a surface with the fungal spray of the subject invention or administering to the mosquito a fungal extract/metabolite or a composition of the subject invention.

In one embodiment, the methods provided herein may require exposing the mosquito to the fungal extract, fungal metabolite, or the composition comprising the fungal extract or fungal metabolite multiple times to, for example, inhibit, reduce or prevent the infection of the mosquito, the malaria transmission, and/or the interaction of malaria parasite and a mosquito.

In one embodiment, each exposure time may be, for example, at least 1 min, at least 5 min, at least 10 min, at least 20 min, at least 30 min, at least 40 min, at least 50 min, at least 60 min, at least 70 min, at least 80 min, at least 90 min, at least 100 min, at least 120 min, at least 150 min, at least 180 min, at least 240 min, at least 300 min, at least 6 hours, at least 8 hours, at least 12 hours, at least 18 hours, at least 24 hours, at least 30 hours, at least 36 hours, at least 42 hours, at least 48 hours, or any time period therebetween.

Also provided is a method for inhibiting the proliferation of the asexual-stage malaria parasite, such as P. falciparum, by using the fungal extracts, fungal metabolite, or the composition comprising the fungal extracts and/or fungal metabolite.

In one embodiment, the concentration of the fungal extract/metabolite may be for example, at least about 0.01 μg/ml; about 0.1 μg/ml, about 1 μg/ml, about 5 μg/ml, about 10 μg/ml, about 20 μg/ml, about 50 μg/ml, or about 100 μg/ml. In one embodiment, the concentration of the fungal extract/metabolite may be for example, from about 0.01 μg/ml to about 500 μg/ml, from about 0.05 μg/ml to about 250 μg/ml, from about 0.1 μg/ml to about 200 μg/ml, from about 0.5 μg/ml to about 100 μg/ml, from about 1 μg/ml to about 100 μg/ml, or from about 2 μg/ml to about 50 μg/ml.

In one embodiment, the inhibition is at least about 5%, at least about 10%, at least about 15%, at least about 20%, at least about 25%, at least about 30%, at least about 35%, at least about 40%, at least about 45, at least about 50%, at least about 55%, at least about 60%, at least about 65%, at least about 70%, at least about 75%, at least about 80%, at least about 85%, at least about 90%, at least about 95%, at least about 99%, at least about 99.5%, or any percentage in between. In a preferred embodiment, the inhibition is 99.9% or 100%.

Further, the subject invention provides a kit comprising fungal extracts, fungal metabolite, a bioactive agent isolated from the fungal extracts, and/or a composition comprising fungal extracts, fungal metabolite, the bioactive agent isolated from the fungal extracts, and optionally, a container containing fungal extracts, fungal metabolite, a bioactive agent isolated from the fungal extracts, and/or the composition. The kit may also comprise a suitable solvent, carrier, vehicle and/or excipient. The kit may further comprise an instruction of using each component.

In one embodiment, the fungal extracts, fungal metabolite, a bioactive agent isolated from the fungal extracts, and/or a composition comprising fungal extracts, fungal metabolite, the bioactive agent isolated from the fungal extracts, is in a dry form such as a solid or powder. In another embodiment, the fungal extracts, fungal metabolite, a bioactive agent isolated from the fungal extracts, and/or a composition comprising fungal extracts, fungal metabolite, the bioactive agent isolated from the fungal extracts, has been dissolved in a suitable solvent, carrier, vehicle and/or excipient.

When ranges are used herein, such as for dose ranges, percentage, combinations and subcombinations of ranges (e.g., subranges within the disclosed range), specific embodiments therein are intended to be explicitly included.

As used herein, the singular forms “a,” “an,” and “the” are intended to include the plural forms as well, unless the context clearly indicates otherwise. Furthermore, to the extent that the terms “including,” “includes,” “having,” “has,” “with,” or variants thereof are used in either the detailed description and/or the claims, such terms are intended to be inclusive in a manner similar to the term “comprising.” The transitional terms/phrases (and any grammatical variations thereof) “comprising,” “comprises,” and “comprise” can be used interchangeably; “consisting essentially of,” and “consists essentially of” can be used interchangeably; and “consisting,” and “consists” can be used interchangeably.

The transitional term “comprising,” “comprises,” or “comprise” is inclusive or open-ended and does not exclude additional, unrecited elements or method steps. By contrast, the transitional phrase “consisting of” excludes any element, step, or ingredient not specified in the claim. The phrases “consisting” or “consists essentially of” indicate that the claim encompasses embodiments containing the specified materials or steps and those that do not materially affect the basic and novel characteristic(s) of the claim. Use of the term “comprising” contemplates other embodiments that “consist” or “consisting essentially of” the recited component(s).

The term “about” or “approximately” means within an acceptable error range for the particular value as determined by one of ordinary skill in the art, which will depend in part on how the value is measured or determined, i.e., the limitations of the measurement system. For example, “about” can mean within 1 or more than 1 standard deviation, per the practice in the art. Alternatively, “about” can mean a range of up to 0-20%, 0 to 10%, 0 to 5%, or up to 1% of a given value. Where particular values are described in the application and claims, unless otherwise stated the term “about” meaning within an acceptable error range for the particular value should be assumed.

Unless otherwise defined, all terms of art, notations and other scientific terms or terminology used herein are intended to have the meanings commonly understood by those of skill in the art to which this invention pertains. In some cases, terms with commonly understood meanings are defined herein for clarity and/or for ready reference, and the inclusion of such definitions herein should not necessarily be construed to represent a substantial difference over what is generally understood in the art. It will be further understood that terms, such as those defined in commonly used dictionaries, should be interpreted as having a meaning that is consistent with their meaning in the context of the relevant art and/or as otherwise defined herein.

### EXAMPLES

### Methods

### Screening the Fungal Extract Library to Discover Malaria Transmission-Blocking Candidates

Small molecules that inhibited the FREP1-Plasmodium interaction to block malaria transmission were screened. In brief, P. falciparum-infected (NF54 obtained from the BEI Resources, Manassas, Va., USA) red blood cells (iRBCs) were cultured for 15-17 days. The iRBCs suspended in PBST (PBS containing 0.2% Tween-20) was homogenized by ultra-sonication with six cycles of 10 s of pulse and 50 s of resting on ice for each period. The lysates were centrifuged at 8,000×g for 2 min to remove insoluble materials and cellular debris. Then, 96-well ELISA plates were coated with 50 μL of the iRBC lysate (2 mg/mL protein) overnight at 4° C. After coating, the wells were blocked with 100 μL of 2% bovine serum albumin (BSA) in PBS per well for 1.5 h at room temperature (RT). After removal of the blocking solution, 49 μL of FREP1 (10 μg/mL) in blocking buffer (PBS containing 2% BSA) and 1 μL of fungal extract (2 mg/mL in DMSO) were added to each well, followed by incubation for 1 h at RT. The wells were washed with 100 μL PBST three times, and 50 μL of rabbit anti-FREP1 polyclonal antibody (1:5,000 dilution in blocking buffer, ˜1 μg/mL as the final concentration) was added to each well and incubated for 1 h at RT. About 50 μL of alkaline phosphatase-conjugated anti-rabbit IgG (Sigma-Aldrich, St Luis, Mo., USA; diluted 1:20,000 in blocking buffer) was added to each well and incubated for 45 min at RT. The wells were washed three times with 100 μL PBST between incubations. Finally, each well was developed with 50 μL of pNPP substrate (Sigma-Aldrich) until the colors were visible, and absorbance at 405 nm was measured. The active recombinant FREP1 supplemented with 1 μL of DMSO was the noninhibition control, and the heat-inactivated recombinant FREP1 (65° C. for 15 min) was the negative control. The following equation was used to calculate the inhibition rate of FREP1-parasite interaction: (A₄₀₅ of DMSO−A₄₀₅ of experimental treatment)/(A₄₀₅ of DMSO−A₄₀₅ of inactivated FREP1).

### Determination of the Activity of Pulixin in Limiting FREP1-Parasite Interaction

The 15-17-day cultured P. falciparum infected cell lysate was prepared as described above. The 96-well ELISA plates were coated with 50 μL of the iRBC lysate (2 mg/mL protein) overnight at 4° C., and the FREP1 protein in PBS (10 μg/mL), together with 0, 2.5, 5, or 10 μg/mL of pulixin, was added to wells and incubated. Rabbit anti-FREP1 polyclonal antibodies quantified the retained FREP1 as described above. After reaction with the pNPP, A₄₀₅ was measured. The assays were conducted in triplicates at each concentration, and the experiments were conducted twice independently.

### Determination of the Transmission-Blocking Activity of the Fungal Extracts and Pure Pulixin

The 15-17-day-old cultured P. falciparum iRBCs containing 2-3% stage V gametocytes were collected and diluted with new O+ type human blood, with the same volume of heat-inactivated AB+ human serum added. The final concentration of stage V gametocytes in the blood was around 0.2%. Then, 3 μL of the candidate fungal extract or pulixin with different concentrations in DMSO was mixed with 297 μL of infected blood and was used to feed about 100 3-5-day-old An. gambiae G3 female mosquitoes for 30 min, and the engorged mosquitoes were maintained with 8% sugar in a BSL-2 insectary (28° C., 12 h light/dark cycle, 80% humidity). The midguts were dissected 7 days after infection and stained with 0.1% mercury dibromofluorescein disodium salt in PBS. The oocysts were counted under a light microscope.

### Determination of Fungal Species

The nuclear ribosomal internal transcribed spacer (ITS) region was amplified with ITS1F (5′-CTTGGTCATTTAGAGGAAGTAA-3′, SEQ ID NO: 1) and ITS4 (5′-TCCTCCGCTTATTGATATGC-3′, SEQ ID NO: 2) primers using the following approach: initial denaturation at 94° C. for 2 min, 35 cycles of denaturation at 94° C. for 30 s, annealing at 55° C. for 30 s, and extension at 72° C. for 1 min, and followed by final extension at 72° C. for 5 min. The amplified product was sequenced with the Sanger approach. Original sequences were searched against GenBank using BLAST to determine the fungal species. Alignments for the ITS locus were carried out in MAFFT v7.307 online version and checked visually and modified manually. A maximum parsimony analysis was performed in PAUP* version 4.0b10. The morphology of the fungi was examined under a microscope (Nikon, Tokyo, Japan). Colonies for observation were grown on potato dextrose agar (PDA) medium plates for 7-15 days at 25° C.

### Extraction, Isolation, and Purification of Active Antimalarial Drug Candidates

About 500 g Cheerios breakfast cereals (General Mills, Minneapolis, Minn.) on an open tray were autoclaved with a cycle of 20 min sterilization and 30 min dry time. The sterile cereals were put into in a mushroom bag. Two liters of sterile 0.3% sucrose solution containing 0.005% chloramphenicol were added, followed by the inoculation of the candidate fungus. The fungus was cultured at room temperature (RT) for 18 days, and then soaked in the same volume of ethyl acetate overnight. The supernatant was filtered using a Büchner funnel and dried using a rotary evaporator (Heidolph, Elk Grove Village, Ill., USA).

The crude extract in methanol were applied onto preparative 60*100 mm-GF254 silica gel thin layer chromatography (TLC) plates (Kaibang Separation Materials LLC, Qingdao, China), separated with the methanol/dichloromethane mixture (1:9 by v/v), and detected the fluorescence bands at 365 nm and the absorbance bands at 254 nm by using the Vis-UV chromatogram analyzer (YUSHEN Instrument Co., Ltd, Shanghai, China). Each band was cut and extracted using 100% methanol. The fractions were dried completely by using a rotary evaporator followed by a vacuum oven. The fractions dissolved in DMSO were analyzed by SMFA for their transmission-blocking activity. The active fraction was subject to Shimadzu HPLC system that included LC-20AD pump, an SPD-20A UV-Vis detector, and an FRC-10A fraction collector (Columbia, Md., USA) with a Gemini column (5 μm C18 110 Å, 250 mm×10 mm, Phenomenex, Torrance, Calif., USA) to purify and evaluate the purity and characterization of the compound using a gradient solvent of methanol-H₂O (50:50-100:0).

### Characterization of Chemical Constituents and Structure

The structure of pulixin was determined by X-ray crystallography. Colorless crystals were obtained by slow evaporation of pulixin in the DMSO solution. Single-crystal X-ray data were collected at 295 K using Mo-Ka radiation on a Bruker D8 Quest diffractometer equipped with a CMOS detector. The structure was confirmed by spectroscopic methods, e.g., ¹H NMR, ¹³C NMR, and ESI-MS. ¹H NMR spectra were recorded on a Bruker-NMR (400 MHz) spectrometer (Bruker Scientific LLC, Billerica, Mass., USA) in DMSO-d6, with 2.5 parts per million (ppm) as the solvent chemical shift. ¹³C NMR spectra were recorded on a Bruker-NMR (100 MHz) spectrometer (Bruker Scientific LLC) in DMSO-d₆, with 39.5 ppm as the solvent chemical shift. Chemical shifts (6) were reported in ppm referenced to the DMSO-d₆ solvent peak. The high-resolution mass spectra (HRMS) were recorded using the (+) ESI mode on a Bruker Daltonics, Impact II QTOF mass spectrometer (gas temperature 200° C.; drying gas (N₂) in a 4 L/min nebulizer at 0.3 bar) at the Mass Spectrometry Research and Education Center of the University of Florida.

### High-Resolution Liquid Chromatography-Mass Spectrometry (LC-MS) Analysis

One milligram of the candidate compound was dissolved in 2 mL of methanol. About 3 μL of this solution was injected with a Dionex UltiMate 3000 Autosampler into a 300 μm×15 cm HPLC C18 column (2 μm, 100 Å Acclaim PepMap; Thermo Fisher Scientific). The HPLC system was the Dionex UltiMate 3000 RSLC nanosystem. The mobile phase was water with 0.1% formic acid (A) and methanol (B). The flow rate of the loading pump was 25 μL/m, and of the NC pump was 5 μL/m. The gradient was 5% B initially, reaching 99% B at 35-45 min, 90% at 45-50 min, and 5% B at 55-60 min. The mass spectrometry data were analyzed with Bruker Daltonics, Impact II QTOF (in positive mode). The gas temperature was 200° C. The drying gas was nitrogen with a flow rate of 4 L/min. The nebulizer was at 0.3 bar.

### Rearing Mosquitoes

An. gambiae (G3 strain) eggs were obtained from BEI Resources (Manassas, Va., USA). Mosquitoes in the insectary was kept at 27° C., 80% relative humidity, and 12 h day/night cycles. The larvae were fed with the ground fish food and the adult mosquitoes were maintained on 8% sucrose solution.

Culturing of P. falciparum Gametocytes and Ookinetes

P. falciparum (NF54) was cultured in the complete RPMI-1640 medium containing 4% new O+ human red blood cells, 10% human AB+ serum, and 12.5 μg/mL of hypoxanthine in a candle jar at 37° C. To prepare P. falciparum ookinetes. 5 mL of day-15 cultured P. falciparum containing ˜2% stage V gametocytes were transferred into a 15 mL centrifuge tube and centrifuged at 650×g for 5 min at RT. The pellet was then resuspended in 500 μL of sterile ookinete culture medium (RPMI-1640 medium containing 20% human serum AB+, 50 μg/mL of hypoxanthine, 2 g/L NaHCO₃). The resuspended cells were transferred into a well of a 12-well plate and incubated at room temperature on a shaker (20 rpm) for 24 h to generate ookinetes. Finally, cell mixtures of the ookinetes, gametocytes, and asexual-stage P. falciparum were collected by centrifugation at 650×g for 5 min at RT.

Analysis of the Conversion Ratio from Gametocytes to Ookinetes

The 15-17-day-old cultured P. falciparum was collected by centrifugation at 500×g for 3 min. The pellets were suspended in ookinete culture medium (incomplete RPMI-1640 containing 20% human serum AB+, 50 μg/mL of hypoxanthine, and 2 g/L of NaHCO₃) to obtain 10⁵ gametocytes per μL. About 1 μL of pulixin (4 mM) in DMSO was added to 99 μL of the ookinete culture medium. After incubation on a shaker (20 rpm) at RT for 18-24 h, the ookinetes and gametocytes were counted using a Giemsa-stained blood smear under a bright-field microscope. The ratios of gametocytes to ookinetes were calculated.

Inhibition Assays of Asexual Plasmodium falciparum Proliferation

The 3-5-day cultured iRBC were mixed with fresh human RBCs (AB+ type) in complete RPMI-1640 to prepare cultures with 0.5% parasitemia and 2% hematocrit. Pulixin was dissolved in DMSO at the concentration of 1 mg/mL and diluted with DMSO to various levels. A 2 μL pulixin solution mixed with 1 mL of cell culture was added to a 24-well plate. The plate was incubated in a candle jar at 37° C. Approximately 48 h later, the medium was replaced with fresh medium containing same concentration of pulixin. Parasitemia was recorded at 24, 48, 72, and 96 h post-incubation. The test for each concentration was replicated three times. EC₅₀ was determined by analyzing the dose-response curve obtained with GraphPad Prism (GraphPad Software, CA, USA). The assays were repeated.

### General Cytotoxicity Assay

A drug could kill a cell or inhibit the cell proliferation through general cytotoxicity. Vybrant® MTT (3-(4,5-dimethylthiazol-2-yl)-2,5-diphenyl tetrazolium bromide) Cell Proliferation Assay (Thermo Fisher) was used to analyze living cells. The human embryonic kidney 293 (HEK293) cell line was used as the experimental cells. About 20,000 HEK293 cells in 100 μL of culture medium (RPMI 1640+2 mM glutamine+10% fetal bovine serum) were seeded per well in 96-well microplates. After incubation at 37° C. with 5% CO2 for 24 h, 1 μL of pulixin in DMSO at various dilutions was added into each well to obtain a final concentration of 0, 1, 3, 10, 30, and 100 μg/mL. Three replicates were conducted for each concentration. Following the incubation at 37° C. with 5% CO2 for 24 h, 100 μL of MTT (5 mg/mL in PBS) was added into each well and incubated for 4 h at 37° C. with 5% CO2. All but 25 μL of the medium was removed from the wells, 100 μL of DMSO was added to each well and incubated at 37° C. for 10 min to dissolve formazan crystals for measurement. Optical density was measured at an absorbance wavelength of 540 nm. The data were analyzed using ANOVA in Prism 8 (GraphPad, San Diego, Calif.). The experiment was independently performed twice.

### Example 1—Bioactive Fungal Extracts Against P. falciparum Transmission

Because FREP1-parasite interaction facilitates malaria transmission, an ELISA-based approach was used to screen 1232 ethyl acetate extracts (40 μg/mL) in the global fungal extract library (GFEL) that prevented FREP1 protein from binding to P. falciparum-infected cell lysate. The extracts that inhibited 90% of the FREP1-parasite interaction were further analyzed for their activities in blocking malaria transmission with SMFA.

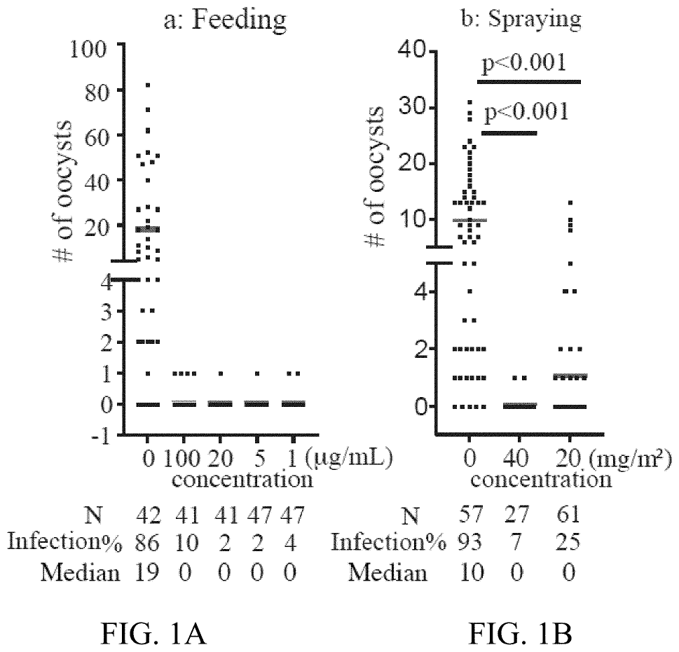

Here, the focus was on one fungal extract, GFEL-12E6 (GFEL plate 12, row E, column 6), because it completely inhibited transmission of P. falciparum to An. gambiae at 1 μg/mL and was more active than the other candidates. A series of dilutions, from 100 μg/mL to 1 μg/mL, of the GFEL-12E6 crude fungal extract inhibited the transmission of P. falciparum to An. gambiae (FIG. 1A). This fungal extract at 1 μg/mL rendered 45 out of 47 mosquitoes free of P. falciparum infection, and 2 out of 47 mosquitoes had only one oocyst. In the control group, about 86% of mosquitoes were infected with P. falciparum, e.g., 32 out of 42 mosquitoes had oocysts in their midguts.

Spraying agents to block malaria transmission is a novel approach. This method will significantly facilitate the future application of antimalarial agents. The effect of GFEL-12E6 sprays on malaria transmission to mosquitoes was examined. The GFEL-12E6 extract in acetone was sprayed on the inner surface of paper cups. After drying, about 100 mosquitoes were placed in the treated cups for 24 h and then fed with P. falciparum-infected blood. The engorged mosquitoes were maintained in a new clean cup without any fungal extract spray. The negative controls were cups treated with acetone only.

Results showed that significantly fewer P. falciparum oocysts were developed in the mosquitoes pre-exposed to GFEL-12E6 than in those in the control (FIG. 1B). Spraying with the fungal extract inhibited P. falciparum infection in mosquitoes. As little as 20 mg/m² of GFEL-12E6 was capable of significantly reducing (p<0.001) P. falciparum infection load in mosquitoes. The median number of oocysts and the infection prevalence rate were 10 and 93%, respectively, in the control group. After exposure to GFEL-12E6 extract spray at 20 mg/m², the median number of oocysts was 0 and infection prevalence was 25% (FIG. 1B). This inhibition was dose-dependent. Spraying with the 40 mg/m² extract made 93% of mosquitoes free from P. falciparum infection (FIG. 1B).

### Example 2—Identification of the Candidate Fungal Species

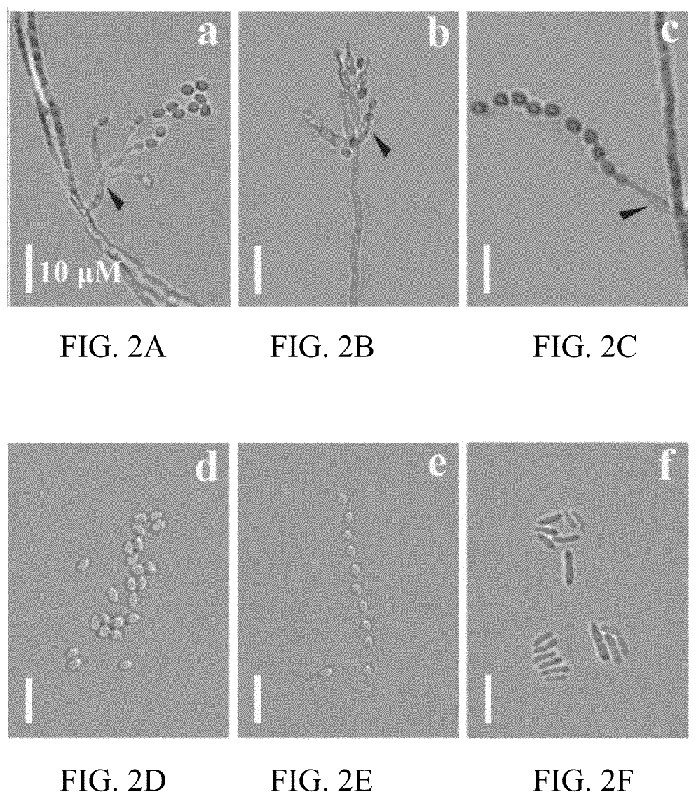

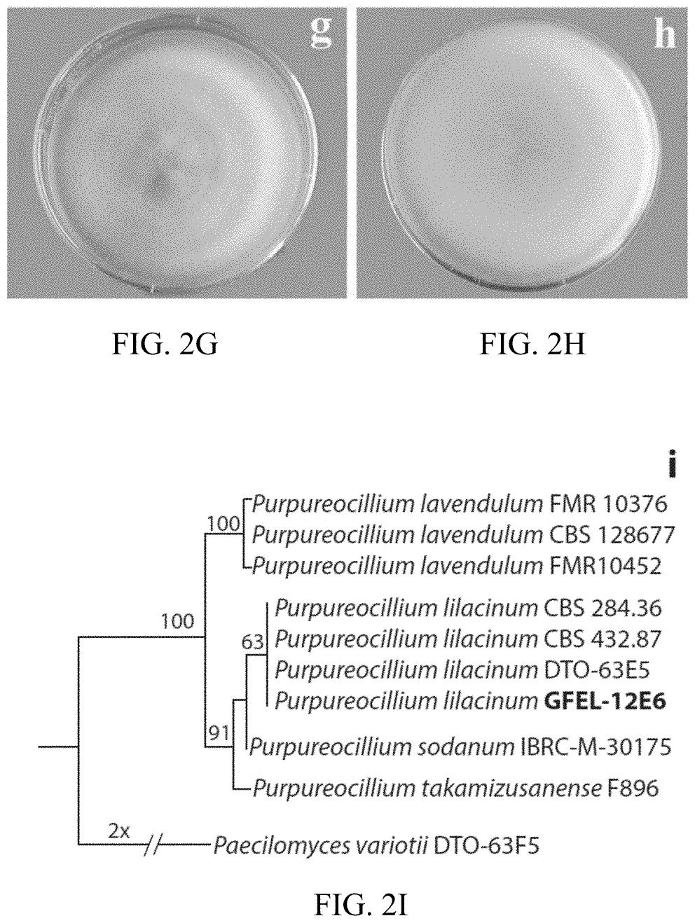

Since GFEL-12E6 is functional in limiting malaria transmission, further studies were undertaken to identify the species of this candidate fungus. The morphology of the candidate fungus was examined under a microscope. The conidiophores growing from the aerial mycelium were short and branched without a specific pattern, with 1-4 phialides per branch (FIG. 2A). In contrast, the conidiophores rising from the superficial mycelium were very long and bore verticillate branches with whorls of 2-4 phialides (FIG. 2B). Phialides were 2.5-3×7-9.5 μm in dimension, with a swollen basal portion tapering into a distinct neck about 1 μm in length (FIGS. 2A, and B). Phialides that produced Acremonium-like conidiophores were very long (up to 30 μm) and solitary (FIG. 2C). Conidia were in long dry chains, subglobose, 2-3×3-4 μm, smooth-walled to slightly roughened, hyaline, and purple in mass (FIGS. 2D, and E). Some conidia were cylindrical and were 1.5-2.5×2.0-13.5 μm in dimension (FIG. 2F). Colonies on potato dextrose agar (PDA) medium plates attained a size of 50 mm and 65 mm in diameter after 15 days and 30 days of incubation, respectively, at 25° C. Colonies consisting of a dense basal felt were white at the beginning, later becoming purple in color (FIG. 2G). From the reverse side of the plate, the colony appeared to be light yellow (FIG. 2H). The morphology of this fungus was similar to that of Purpureocillium lilacinum re-named from Paecilomyces lilacinum.

To further identify the species, the conserved intergenic space of the fungal genome was PCR-amplified with ITS1/ITS4 primers and sequenced. Phylogenetic analysis of the ITS (FIG. 2I) showed that the candidate fungus GFEL-12E6 and other Purpureocillium species were in the same monophyletic Glade with 100% maximum parsimony (MP). The candidate fungus was clustered together with the identified Purpureocillium lilacinum strains (63% MP). The phylogenetic analysis confirmed that the candidate fungus GFEL-12E6 was Purpureocillium lilacinum.

### Example 3—Isolation and Identification of Fungal Metabolites

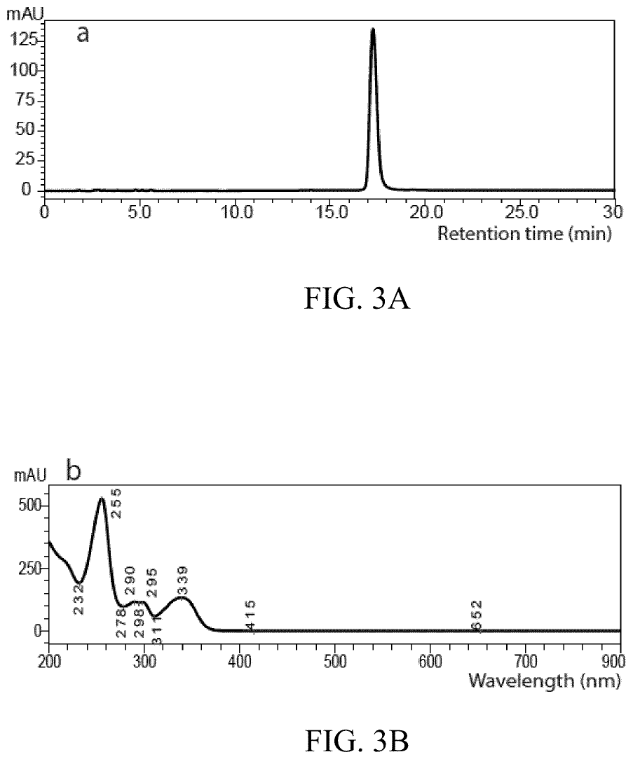

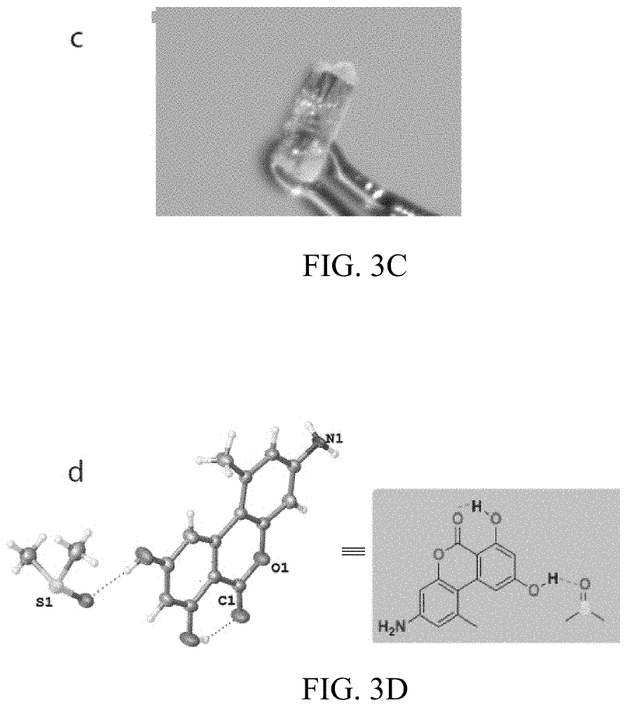

GFEL-12E6 in methanol was fractioned by preparative TLC and the bioactivity of each fraction was detected by SMFA. One bioactive fraction was further purified by HPLC to obtain a pure compound at 17.2 min retention time (FIG. 3A). The active compound was named “pulixin.” UV-visible absorbance spectra showed characteristic peaks at 255, 290, 295, and 339 nm (FIG. 3B).

Furthermore, pulixin was crystallized from DMSO by slow evaporation at RT (FIG. 3C), yielding a colorless solid crystal. The structure of the crystal was determined by X-ray crystallography. In the crystal, an interstitial DMSO molecule was hydrogen-bonded to pulixin. Based on the X-ray structure determination, pulixin was identified as 3-amino-7,9-dihydroxy-1-methyl-6H-benzo[c]chromen-6-one (FIG. 3D). The crystal data were submitted to the Cambridge Crystallographic Data Centre with the deposition number 2005130.

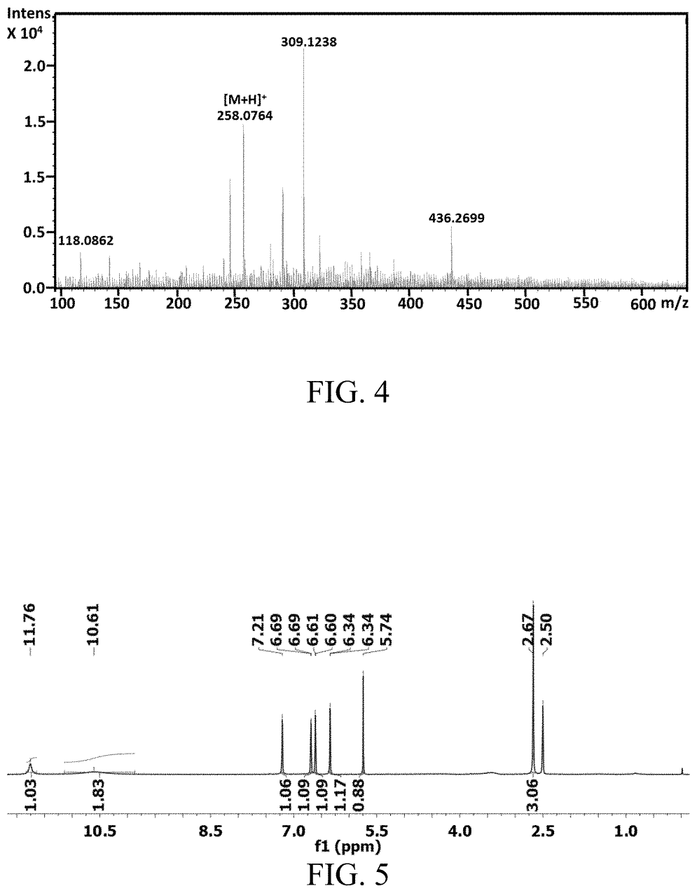

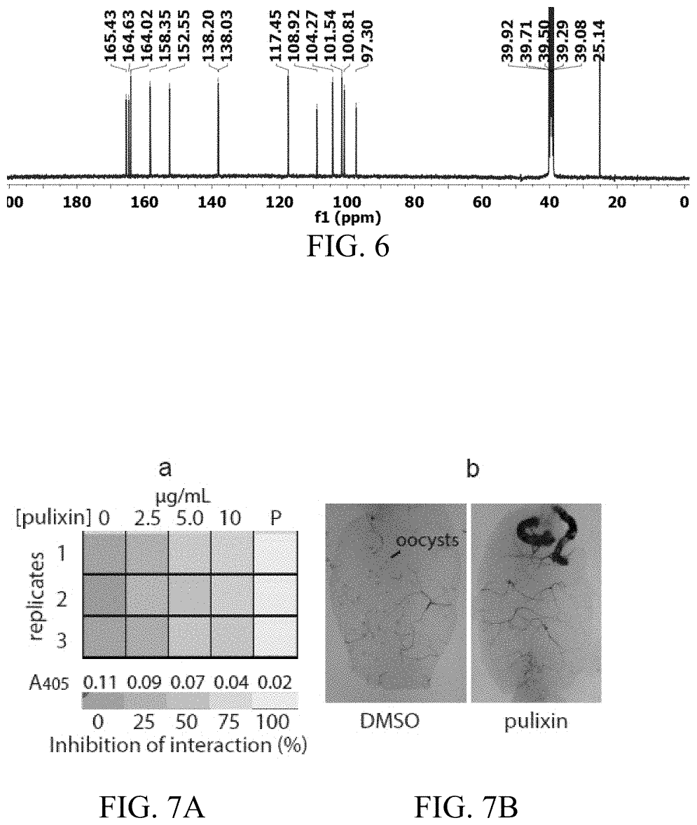

The molecular mass of pulixin was also determined using HR-ESI-mass spectrometry to confirm its identity in the bulk extract. An [M+H]⁺ ion at m/z 258.0764 was observed, matching the calculated mass of 258.0766 amu (FIG. 4). In addition, NMR was used to confirm the structure of the active compound. ¹H-NMR data (FIG. 5) confirmed the presence of a specific hydrogen-bonded hydroxyl group (δ=11.76 ppm), an amino group (δ=10.61 ppm), and a methyl group (δ=2.67 ppm). The ¹³C-NMR data (FIG. 6) were consistent with the presence of an ester keto group (δ=165.4 ppm), two hydroxyl groups bearing aromatic carbon atoms (δ=164.6 and 164.0 ppm), one amino group attached to aromatic carbon atom (δ=152.6 ppm), and a methyl carbon atom attached to the aromatic ring (δ=25.2 ppm). Table 1 summarizes the NMR data. Collectively, the data for the structure of pulixin unambiguously confirmed it to be 3-amino-7,9-dihydroxy-1-methyl-6H-benzo[c]chromen-6-one.

TABLE 1
¹H NMR and ¹³C NMR data of antimalarial drug 3-Amino-7,9-dihydroxy-1-methyl-
6Hbenzo[c]chromen-6-one (δ in ppm, J in Hz and NMR Solvent DMSO-d₆).

| Position | ¹H NMR data (400 MHz) | ¹³C NMR data (100 MHz) | Structure |
|---|---|---|---|
| 1 2 3 4 5 6 7 8 9 10 11 12 13 | — 6.69 (d, J = 2.1 Hz, 1H) — 6.61 (d, J = 2.2 Hz, 1H) — — — 6.34 (d, J = 1.2 Hz, 1H) — 7.21 (s, 1H) — — — | 138.2 108.9 152.6 100.8 158.4 101.6 164.6 97.3 164.0 104.3 138.0 117.5 165.4 | 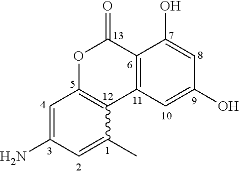 [SMILES: Cc1cc(N)cc2oc(=O)c3c(O)cc(O)cc3c12] 1 |
| —CH₃ | 2.67 (s, 3H) | 25.1 | |
| —NH₂ | 10.61 (s, br, 2H) | | |
| —OH | 11.76 (s, 1H), 5.74 (s, 1H) | | |

### Example 4—Pulixin Prevented FREP1 from Binding to P. Falciparum and Blocked P. Falciparum Transmission to An. gambiae

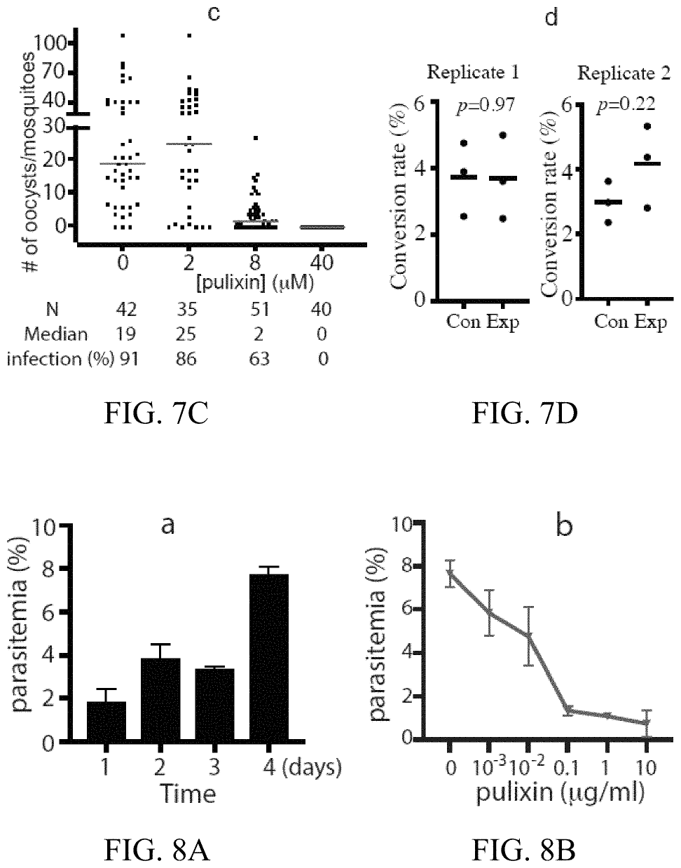

The activity of pulixin in preventing FREP1 from binding to parasite-infected cell lysate was determined using ELISA assays. The A405 values differed among wells with different concentrations of pulixin (FIG. 7A). As the pulixin concentration increased from 0 to 10 μg/mL, less FREP1 was retained. DMSO (1%, v/v) without the compound was used as a non-inhibition control. The heat-inactivated FREP1, which did not bind to the parasite-infected cell lysate, was used to replace FREP1 as the 100% inhibition control (labeled as P in FIG. 7A). Based on the A₄₀₅ values, inhibition rates at different concentrations were calculated. The results showed that pulixin inhibited the interaction between the FREP1 protein and P. falciparum-infected cell lysate, and the inhibition was dose-dependent (FIG. 7A). At a concentration of 5 μg/mL, pulixin inhibited about 50% of the interaction between the FREP1 protein and P. falciparum-infected cell lysate.

Next, the effects of pulixin on P. falciparum infection in mosquitoes were analyzed. Pure pulixin in DMSO was mixed with P. falciparum-infected blood at concentrations from 0 μM to 40 μM and fed to An. gambiae using SMFA. The midguts in the experimental groups contained fewer oocysts, stained and shown as dots, than those in the control (DMSO; FIG. 7B). Pulixin completely inhibited malaria transmission at a concentration of 40 μM, and inhibition activity decreased as the level of pulixin decreased (FIG. 7C). EC₅₀, defined as the concentration of a compound that inhibits 50% of infection intensity (the number of oocysts per mosquito), in experimental mosquitoes compared to that of the control group was 11 μM, calculated using a serial dilution of samples with an LC₅₀ calculator. The activity of pulixin was analyzed again after storing it in the laboratory at RT for six months and obtained a similar result.

Pulixin at a level of 40 μM completely blocked malaria transmission. Therefore, it was examined whether 40 μM of pulixin inhibited conversion of gametocytes to ookinetes. Results showed no significant difference in gametocyte transformation rates between the control and pulixin-treated samples (p>0.2; FIG. 4d), supporting that pulixin did not affect the conversion of gametocytes to ookinetes.

### Example 5—Pulixin Inhibited the Development of the Asexual P. falciparum

First, the parasitemia was analyzed to determine the parasite proliferation profile in four days. P. falciparum infected blood was added into the fresh medium with uninfected red blood cells to obtain 0.5% parasitemia in 2% hematocrit. The culture was incubated for 4 days with medium changed on day 2 and the parasitemia was analyzed every day.

Results (FIG. 8A) showed that parasitemia on day 4 is significantly higher (p<0.001) than day 1 and almost all infected cells were at asexual stage. This result is consistent to the P. falciparum 48-hour asexual replication cycle. Based on this result, the activity of pulixin was examined in inhibiting the development of asexual-stage P. falciparum on 4th day after inoculation. The results showed that the inhibitory effect of pulixin on asexual-stage P. falciparum development was dose-dependent and inhibition increased as the pulixin concentration increased (FIG. 8B). The EC₅₀ of pulixin in inhibiting the development of the asexual-stage P. falciparum was 0.012 μg/mL or 47 nM.

### Example 6—Pulixin Did not Show General Cytotoxicity to Human Cells

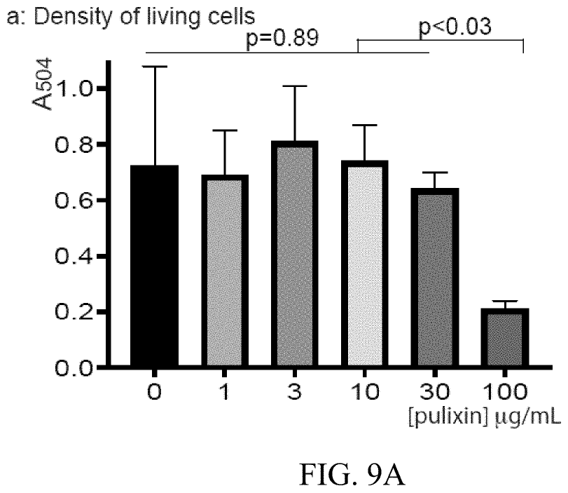

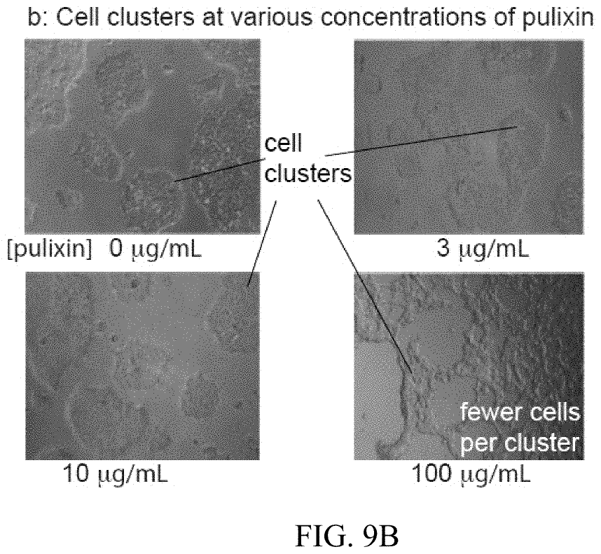

Following confirmation that pulixin inhibited both the sexual and asexual stages of P. falciparum, its general cytotoxicity to cells was analyzed using MTT assays. These assays measured the density of living cells. Human embryonic kidney 293 cells (HEK293) were incubated with pulixin at different concentrations. No significant difference in the density of living cells was observed in cultures with a pulixin level ranging from 0 to 30 μg/mL (116 μM; p=0.88; FIG. 9A) suggesting that pulixin did not have significant cytotoxic effects on HEK293 cells at these concentrations. When the level of pulixin increased to 100 μg/mL, a significant reduction in cell density occurred (p<0.05; FIG. 9A) and much fewer cells per cluster were observed under a microscope (FIG. 9B) compared to the other groups.

All patents, patent applications, provisional applications, and publications referred to or cited herein are incorporated by reference in their entirety, including all figures and tables, to the extent they are not inconsistent with the explicit teachings of this specification.

It should be understood that the examples and embodiments described herein are for illustrative purposes only and that various modifications or changes in light thereof will be suggested to persons skilled in the art and are to be included within the spirit and purview of this application and the scope of the appended claims. These examples should not be construed as limiting. In addition, any elements or limitations of any invention or embodiment thereof disclosed herein can be combined with any and/or all other elements or limitations (individually or in any combination) or any other invention or embodiment thereof disclosed herein, and all such combinations are contemplated within the scope of the invention without limitation thereto.

We claim:

1. A composition comprising a fungal extract and a pharmaceutically acceptable carrier, the fungal extract being a Purpureocillium lilacinum extract.

2. The composition of claim 1, further comprises a fungal extract selected from Penicillium thomii, Penicillium pancosmium, Aspergillus niger, and Aspergillus aculeatus.

3. The composition of claim 1, the fungal extract comprising a bioactive fungal metabolite having a general structure of formula (I): 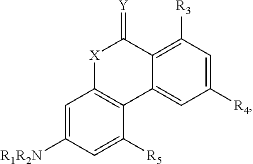
wherein X and Y are independently selected from S, N and O; R₁ and R₂ are independently selected from hydrogen, alkyl and substituted alkyl; and R₃, R₄ and R₅ are independently selected from hydrogen, alkyl, substituted alkyl, —NR₁R₂, and —OR₆, wherein R₆ is hydrogen, alkyl, aryl, substituted alkyl or substituted aryl.

4. The composition of claim 3, the fungal metabolite being pulixin.

5. The composition of claim 1, further comprises asperaculane B, and/or P-orlandin.

6. The composition of claim 1, the fungal extract being a hexane, dichloromethane, ethanol, methanol, ethyl acetate, acetone, or acetyl acetate extract.

7. The composition of claim 1, which is formulated as a spray.

8. The composition of claim 1, the fungal extract being in a solid, semi-solid or powder form.

9. A method of inhibiting malaria infection in a subject in need thereof comprising administering the composition of claim 1 to the subject.

10. The method of claim 9, the malaria is caused by P. falciparum, P. malariae, P. ovale, P. vivax, P. knowlesi, P. berghei, P. chabaudi and P. yoelii.

11. The method of claim 9, the administration being oral, nasal, topical, transdermal, or parenteral.

12. A method of inhibiting malaria transmission to a mosquito, the method comprising exposing the mosquito to the composition of claim 1.

13. The method of claim 12, the malaria is caused by P. falciparum, P. malariae, P. ovale, P. vivax, P. knowlesi, P. berghei, P. chabaudi and P. yoelii.

14. The method of claim 12, the exposing comprising contacting/feeding the mosquito or spraying a surface where the mosquito is sitting or landing.

15. The method of claim 14, the surface being human skin, wall surface, floor surface, and a surface of a furniture.

16. A method of inhibiting the interaction of malaria parasite and a mosquito, the method comprising exposing the mosquito to the composition of claim 1.

17. The method of claim 16, the exposing comprising contacting/feeding the mosquito or spraying a surface where the mosquito is sitting or landing.

18. The method of claim 17, the surface being human skin, wall surface, floor surface, and a surface of a furniture.

19. A method of inhibiting the interaction of malaria parasite and a midgut protein of a mosquito, the method comprising exposing the mosquito to the composition of claim 1.

20. The method of claim 19, the midgut protein being FREP1.


SEQUENCE LISTING (2 sequences)

SEQ ID NO: 1 - DNA, 22 bp, Artificial sequence
  primer
cttggtcatttagaggaagtaa

SEQ ID NO: 2 - DNA, 20 bp, Artificial sequence
  primer
tcctccgcttattgatatgc